(19) United States
(12) Patent Application Publication
(10) Pub. No.: US 2023/0241064 A1
(43) Pub. Date: Aug. 3, 2023

(54) METHODS OF TREATMENT AND/OR PREVENTION OF MAJOR ADVERSE CARDIOVASCULAR EVENTS (MACE) WITH A COMBINATION OF A BET BROMODOMAIN INHIBITOR AND A DIPEPTIDYL PEPTIDASE 4 INHIBITOR

(71) Applicant: Resverlogix Corp., Calgary (CA)

(72) Inventors: Kenneth Eugene LEBIODA, Calgary (CA); Christopher Ross Armstrong HALLIDAY, Calgary (CA); Aziz Naeem KHAN, Calgary (CA)

(21) Appl. No.: 17/791,700

(22) PCT Filed: Jan. 7, 2021
(86) PCT No.: PCT/IB2021/000006

§ 371 Date: Nov. 11, 2022

Related U.S. Application Data
(60) Provisional application No. 62/958,474, filed on Jan. 8, 2020.

(51) Int. Cl. A61K 31/517; A61P 9/10

(52) U.S. Cl. CPC A61K 31/517; A61P 9/10

(57) ABSTRACT

Described herein are methods of for treating and/or preventing Major adverse cardiovascular events (MACE) by administering to a subject in need thereof, a combination of a dipeptidyl peptidase 4 (DPP-4) inhibitor and a compound of Formula I or a stereoisomer, tautomer, pharmaceutically acceptable salt, or hydrate thereof, wherein the variables of Formula I are as defined herein.

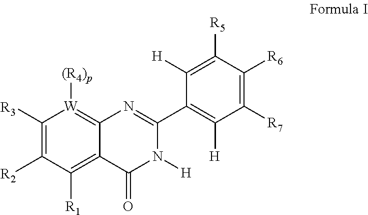

This application claims the benefit of priority of U.S. Provisional Application No. 62/958,474, filed Jan. 8, 2020, the entire disclosure of which is incorporated herein by reference.

The present disclosure relates to methods of treating and/or preventing Major adverse cardiovascular events (MACE) (including non-fatal myocardial infarction, cardiovascular death, stroke, and hospitalization for cardiovascular disease (CVD) events) by administering to a subject in need thereof, a combination of a dipeptidyl peptidase 4 (DPP-4) inhibitor and a compound of Formula I or a stereoisomer, tautomer, pharmaceutically acceptable salt, or hydrate thereof.

Despite the use of modern evidence-based therapies including prompt coronary revascularization, dual anti-platelet therapy, and intensive lipid lowering therapy, major adverse cardiovascular events (MACE) recur with high frequency after an acute coronary syndrome (ACS). Patients with type 2 diabetes (T2DM) have a particular high risk and represents about one third of ACS cases (Cannon et al. 2015; Schwartz et al. 2013; Schwartz et al. 2018). Dipeptidyl peptidase 4 or DPP-4 inhibitors are a class of oral diabetes drugs that inhibit the enzyme DPP-4. These therapies function by inhibiting the degradation of the incretins, glucagon-like peptide-1 (GLP-1) and glucose-dependent insulinotropic peptide (GIP), and therefore could potentially affect glucose regulation through multiple effects (Thornberry and Gallwitz. 2009). DPP-4 inhibition treatment has shown a noninferior risk of cardiovascular-related disorder risk in patients with established cardiovascular disease, diabetes, chronic kidney disease and ACS (Rosenstock et al. 2019; Green et al. 2015; Scirica et al. 2013; White et al. 2013). However, no DPP-4 inhibitor has been shown to reduce MACE in patients with recent ACS, and substantial residual risk remains for this population. At best, DPP-4 inhibitors have been shown to have a neutral effect on MACE in clinical trials that assessed their cardiovascular safety. For example, the SAVOR-TIMI-53 and EXAMINE suggested a neutral effect of saxagliptin and alogliptin on the 3-point MACE composite outcome of cardiovascular death, myocardial infarction, or ischemic stroke. Similarly, in the TECOS trial that assessed the safety of sitagliptin, the DPP-4 inhibitor was found to be noninferior to placebo for both a 3-point [hazard ratio (HR) 0.99; 95% confidence interval (CI) 0.89-1.10) and 4-point (HR 0.98; 95% CI 0.89-1.08) MACE composite outcome (Karagiannis et al. 2015).

Apabetalone (RVX-208 or RVX000222) is a first-in-class Bromodomain and Extra-Terminal (BET)-inhibitor (BETi) that binds selectively to the second bromodomain of BET proteins (e.g., BRD2, BRD3, BRD4 and BRDT) to prevent BET protein translocation and thereby inhibit the transcription of genes that drive chronic diseases. A recently completed clinical Phase 3 trial (BETonMACE; NCT02586155) evaluated the effect on MACE of apabetalone (RVX-208) in type 2 diabetes patients with low HDL cholesterol (below 40 mg/dL for males and below 45 mg/dL for females) and a recent ACS (preceding 7-90 days). All patients received high intensity or maximum-tolerated statin treatment. The study enrolled 2,425 patients and the full analysis set (FAS) population, from which the MACE outcomes were evaluated, consisted of 2,418 patients. A total of 169 patients received both RVX-208 and a DPP-4 inhibitor; a total of 167 received a DPP-4 inhibitor, but no RVX-208; a total of 1,043 received RVX-208, but no DPP-4 inhibitor; a total of 1,039 received neither RVX-208 or a DPP-4 inhibitor.

Surprisingly, as detailed in Example 2, we found that patients treated with the combination RVX-208 and a DPP-4 inhibitor showed pronounced reduction of cardiovascular-related disorders and cardiovascular disease (CVD) events, as measured by MACE reduction, compared to treatment with either therapy alone. As discussed above, no DPP-4 inhibitor has been shown to reduce MACE. The results discussed in Example 2 consistently demonstrate that like DPP-4 inhibitors, apabetalone by itself does not reduce hazard ratios or the number of patients having a MACE event (as a single composite end point of the events non-fatal myocardial infarction, cardiovascular death, stroke and optionally hospitalization for cardiovascular diseases) and the specific MACE events myocardial infarction, cardiovascular death, and hospitalization for cardiovascular diseases (see FIGS. 2, 5, 8, and 11). However, when apabetalone was combined with a DPP-4 inhibitor, the number of patients having a MACE event as a whole or a specific individual MACE event was unexpectedly and consistently reduced to an extent that reached statistical significance (e.g., at least about 30% and up to about 80%; see FIGS. 1, 3, 4, 6, 7, 9, 10, 12, 13, and 15) compared to either apabetalone monotherapy or DPP-4 inhibitor monotherapy.

Accordingly, the technical solution provided by the present disclosure includes methods of treating and/or preventing Major adverse cardiovascular events (MACE) (including non-fatal myocardial infarction, cardiovascular death, stroke, and hospitalization for CVD events) by administering to a subject in need thereof, a dipeptidyl peptidase 4 (DPP-4) inhibitor and a compound of Formula I or a stereoisomer, tautomer, pharmaceutically acceptable salt, or hydrate thereof.

Compounds of Formula I have previously been described in U.S. Pat. No. 8,053,440, incorporated herein by reference. Compounds of Formula I include:

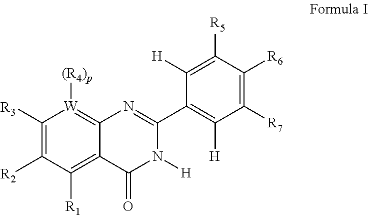

or a stereoisomer, tautomer, pharmaceutically acceptable salt, or hydrate thereof,

wherein:

R₁ and R₃ are each independently selected from alkoxy, alkyl, amino, halogen, and hydrogen;

R₂ is selected from alkoxy, alkyl, alkenyl, alkynyl, amide, amino, halogen, and hydrogen;

R₅ and R₇ are each independently selected from alkyl, alkoxy, amino, halogen, and hydrogen;

R₆ is selected from amino, amide, alkyl, hydrogen, hydroxyl, piperazinyl, and alkoxy;

W is selected from C and N, wherein if W is N, then p is 0 or 1, and if W is C, then p is 1; and

for W—(R₄)ₚ, W is C, p is 1 and R₄ is H, or W is N and p is 0.

Apabetalone (RVX-208 or RVX000222) is a representative example of Formula I.

In some embodiments, the invention provides methods of preventing cardiovascular death by administering to a subject in need thereof, a dipeptidyl peptidase 4 (DPP-4) inhibitor and a compound of Formula I or a stereoisomer, tautomer, pharmaceutically acceptable salt, or hydrate thereof.

In some embodiments, the invention provides methods of treating and/or preventing hospitalization for CVD events by administering to a subject in need thereof, a dipeptidyl peptidase 4 (DPP-4) inhibitor and a compound of Formula I or a stereoisomer, tautomer, pharmaceutically acceptable salt, or hydrate thereof.

In some embodiments, the invention provides methods of treating and/or preventing a non-fatal myocardial infarction by administering to a subject in need thereof, a dipeptidyl peptidase 4 (DPP-4) inhibitor and a compound of Formula I or a stereoisomer, tautomer, pharmaceutically acceptable salt, or hydrate thereof.

In some embodiments, the compound of Formula I is administered simultaneously with a DPP-4 inhibitor. In some embodiments, the compound of Formula I is administered sequentially with the DPP-4 inhibitor. In some embodiments, the compound of Formula I is administered in a single pharmaceutical composition with the DPP-4 inhibitor. In some embodiments, the Compound of Formula I and the DPP-4 inhibitor are administered as separate compositions.

In some embodiments, the compound of Formula Ia is selected from

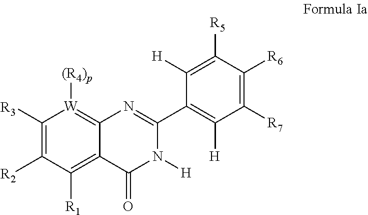

or a stereoisomer, tautomer, pharmaceutically acceptable salt, or hydrate thereof,

wherein:

R₁ and R₃ are each independently selected from alkoxy, alkyl, and hydrogen;

R₂ is selected from alkoxy, alkyl, and hydrogen;

R₅ and R₇ are each independently selected from alkyl, alkoxy, and hydrogen;

R₆ is selected from alkyl, hydroxyl, and alkoxy;

W is selected from C and N, wherein if W is N, then p is 0 or 1, and if W is C, then p is 1; and

for W—(R₄)ₚ, W is C, p is 1 and R₄ is H, or W is N and p is 0.

In some embodiments, the Compound of Formula I is 2-(4-(2-hydroxyethoxy)-3,5-dimethylphenyl)-5,7-dimethoxyquinazolin-4(3H)-one (RVX-208 or RVX000222) or a pharmaceutically acceptable salt thereof.

In some embodiments, the DPP-4 inhibitor is sitagliptin, saxagliptin, linagliptin, alopgliptin, vildagliptin, anagliptin, trelagliptin, omarigliptin, evogliptin, gosogliptin, gemigliptin, teneligliptin, or dutogliptin.

In some embodiments, the MACE endpoint is narrowly defined as a single composite endpoint of cardiovascular (CV) death, non-fatal myocardial infarction, or stroke.

In some embodiments, the MACE endpoint is broadly defined as a single composite endpoint of cardiovascular (CV) death, non-fatal myocardial infarction, hospitalization for CVD events, or stroke. In one embodiment, the CVD is congestive heart failure. In one embodiment, the hospitalization for cardiovascular disease events is hospitalization for congestive heart failure.

### BRIEF DESCRIPTION OF THE DRAWINGS

FIG. 1 depicts a comparison of the cumulative incidence of narrowly defined MACE in patients administered RVX-208 with DPP-4 inhibitors versus patients administered placebo with DPP-4 inhibitors.

FIG. 2 depicts a comparison of the cumulative incidence of narrowly defined MACE in patients administered RVX-208 without DPP-4 inhibitors versus patients administered placebo without DPP-4 inhibitors.

FIG. 3 depicts a comparison of the cumulative incidence of narrowly defined MACE in patients administered RVX-208 with DPP-4 inhibitors versus patients administered RVX-208 without DPP-4 inhibitors.

FIG. 4 depicts a comparison of the cumulative incidence of broadly defined MACE in patients administered RVX-208 with DPP-4 inhibitors versus patients administered placebo with DPP-4 inhibitors.

FIG. 5 depicts a comparison of the cumulative incidence of broadly defined MACE in patients administered RVX-208 without DPP-4 inhibitors versus patients administered placebo without DPP-4 inhibitors.

FIG. 6 depicts a comparison of the cumulative incidence of broadly defined MACE in patients administered RVX-208 with DPP-4 inhibitors versus patients administered RVX-208 without DPP-4 inhibitors.

FIG. 7 depicts a comparison of the cumulative incidence of non-fatal myocardial infarction in patients administered RVX-208 with DPP-4 inhibitors versus patients administered placebo with DPP-4 inhibitors.

FIG. 8 depicts a comparison of the cumulative incidence of non-fatal myocardial infarction in patients administered RVX-208 without DPP-4 inhibitors versus patients administered placebo without DPP-4 inhibitors.

FIG. 9 depicts a comparison of the cumulative incidence of non-fatal myocardial infarction in patients administered RVX-208 with DPP-4 inhibitors versus patients administered RVX-208 without DPP-4 inhibitors.

FIG. 10 depicts a comparison of the cumulative incidence of CV deaths in patients administered RVX-208 with DPP-4 inhibitors versus patients administered placebo with DPP-4 inhibitors.

FIG. 11 depicts a comparison of the cumulative incidence of CV deaths in patients administered RVX-208 without DPP-4 inhibitors versus patients administered placebo without DPP-4 inhibitors.

FIG. 12 depicts a comparison of the cumulative incidence of CV deaths in patients administered RVX-208 with DPP-4 inhibitors versus RVX-208 without DPP-4 inhibitors.

FIG. 13 depicts a comparison of the cumulative incidence of hospitalization for congestive heart failure in patients administered RVX-208 with DPP-4 inhibitors versus patients administered placebo with DPP-4 inhibitors.

FIG. 14 depicts a comparison of the cumulative incidence of hospitalization for congestive heart failure events in patients administered RVX-208 without DPP-4 inhibitors versus patients administered placebo without DPP-4 inhibitors.

FIG. 15 depicts a comparison of the cumulative incidence of hospitalization for congestive heart failure events in patients administered RVX-208 with DPP-4 inhibitors versus patients administered RVX-208 without DPP-4 inhibitors.

### DEFINITIONS

By “optional” or “optionally” is meant that the subsequently described event or circumstance may or may not occur, and that the description includes instances where the event or circumstance occurs and instances in which is does not. For example, “optionally substituted aryl” encompasses both “aryl” and “substituted aryl” as defined below. It will be understood by those skilled in the art, with respect to any group containing one or more substituents, that such groups are not intended to introduce any substitution or substitution patterns that are sterically impractical, synthetically non-feasible and/or inherently unstable.

As used herein, the term “hydrate” refers to a crystal form with either a stoichiometric or non-stoichiometric amount of water is incorporated into the crystal structure.

The term “alkenyl” as used herein refers to an unsaturated straight or branched hydrocarbon having at least one carbon-carbon double bond, such as a straight or branched group of 2-8 carbon atoms, referred to herein as (C₂-C₈) alkenyl. Exemplary alkenyl groups include, but are not limited to, vinyl, allyl, butenyl, pentenyl, hexenyl, butadienyl, pentadienyl, hexadienyl, 2-ethylhexenyl, 2 propyl 2-butenyl, and 4-(2-methyl-3-butene)-pentenyl.

The term “alkoxy” as used herein refers to an alkyl group attached to an oxygen (O-alkyl). “Alkoxy” groups also include an alkenyl group attached to an oxygen (“alkenyloxy”) or an alkynyl group attached to an oxygen (“alkynyloxy”) groups. Exemplary alkoxy groups include, but are not limited to, groups with an alkyl, alkenyl or alkynyl group of 1-8 carbon atoms, referred to herein as (C₁-C₈) alkoxy. Exemplary alkoxy groups include, but are not limited to, methoxy and ethoxy.

The term “alkyl” as used herein refers to a saturated straight or branched hydrocarbon, such as a straight or branched group of 1-8 carbon atoms, referred to herein as (C₁-C₈) alkyl. Exemplary alkyl groups include, but are not limited to, methyl, ethyl, propyl, isopropyl, 2-methyl-1-propyl, 2-methyl-2-propyl, 2-methyl-1-butyl, 3 methyl-1-butyl, 2-methyl-3-butyl, 2,2-dimethyl-1-propyl, 2-methyl-1-pentyl, 3 methyl-1-pentyl, 4-methyl-1-pentyl, 2-methyl-2-pentyl, 3-methyl-2-pentyl, 4 methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, and octyl.

The term “amide” as used herein refers to the form NRₐC(O)(R_{b}) or C(O)NR_{b}R_{c}, wherein Rₐ, R_{b} and R_{c} are each independently selected from alkyl, alkenyl, alkynyl, aryl, arylalkyl, cycloalkyl, haloalkyl, heteroaryl, heterocyclyl, and hydrogen. The amide can be attached to another group through the carbon, the nitrogen, R_{b}, or R_{c}. The amide also may be cyclic, for example R_{b} and R_{c}, may be joined to form a 3- to 8-membered ring, such as 5- or 6-membered ring. The term “amide” encompasses groups such as sulfonamide, urea, ureido, carbamate, carbamic acid, and cyclic versions thereof. The term “amide” also encompasses an amide group attached to a carboxy group, e.g., amide-COOH or salts such as amide-COONa, an amino group attached to a carboxy group (e.g., amino-COOH or salts such as amino-COONa).

The term “amine” or “amino” as used herein refers to the form NR_{d}Rₑ or N(R_{d})Rₑ, where R_{d} and Rₑ are independently selected from alkyl, alkenyl, alkynyl, aryl, arylalkyl, carbamate, cycloalkyl, haloalkyl, heteroaryl, heterocycle, and hydrogen. The amino can be attached to the parent molecular group through the nitrogen. The amino also may be cyclic, for example any two of R_{d} and Rₑ may be joined together or with the N to form a 3- to 12-membered ring (e.g., morpholino or piperidinyl). The term amino also includes the corresponding quaternary ammonium salt of any amino group. Exemplary amino groups include alkylamino groups, wherein at least one of R_{d} and Rₑ is an alkyl group. In some embodiments R_{d} and Rₑ each may be optionally substituted with hydroxyl, halogen, alkoxy, ester, or amino.

The term “aryl” as used herein refers to a mono-, bi-, or other multi carbocyclic, aromatic ring system. The aryl group can optionally be fused to one or more rings selected from aryls, cycloalkyls, and heterocyclyls. The aryl groups of this present disclosure can be substituted with groups selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Exemplary aryl groups also include but are not limited to a monocyclic aromatic ring system, wherein the ring comprises 6 carbon atoms, referred to herein as “(C₆) aryl.”

The term “arylalkyl” as used herein refers to an alkyl group having at least one aryl substituent (e.g., aryl-alkyl). Exemplary arylalkyl groups include, but are not limited to, arylalkyls having a monocyclic aromatic ring system, wherein the ring comprises 6 carbon atoms, referred to herein as “(C₆) arylalkyl.”

The term “carbamate” as used herein refers to the form R_{g}OC(O)N(Rₕ), R_{g}OC(O)N(Rₕ)Rᵢ, or OC(O)NRₕRᵢ, wherein R_{g}, Rₕ and Rᵢ are each independently selected from alkyl, alkenyl, alkynyl, aryl, arylalkyl, cycloalkyl, haloalkyl, heteroaryl, heterocyclyl, and hydrogen. Exemplary carbamates include, but are not limited to, arylcarbamates or heteroaryl carbamates (e.g., wherein at least one of R_{g}, Rₕ and Rᵢ are independently selected from aryl or heteroaryl, such as pyridine, pyridazine, pyrimidine, and pyrazine).

The term “carbocycle” as used herein refers to an aryl or cycloalkyl group.

The term “carboxy” as used herein refers to COOH or its corresponding carboxylate salts (e.g., COONa). The term carboxy also includes “carboxycarbonyl,” e.g. a carboxy group attached to a carbonyl group, e.g., C(O)—COOH or salts, such as C(O)—COONa.

The term “cycloalkoxy” as used herein refers to a cycloalkyl group attached to an oxygen.

The term “cycloalkyl” as used herein refers to a saturated or unsaturated cyclic, bicyclic, or bridged bicyclic hydrocarbon group of 3-12 carbons, or 3-8 carbons, referred to herein as “(C₃-C₈)cycloalkyl,” derived from a cycloalkane. Exemplary cycloalkyl groups include, but are not limited to, cyclohexanes, cyclohexenes, cyclopentanes, and cyclopentenes. Cycloalkyl groups may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Cycloalkyl groups can be fused to other cycloalkyl saturated or unsaturated, aryl, or heterocyclyl groups.

The term “dicarboxylic acid” as used herein refers to a group containing at least two carboxylic acid groups such as saturated and unsaturated hydrocarbon dicarboxylic acids and salts thereof. Exemplary dicarboxylic acids include alkyl dicarboxylic acids. Dicarboxylic acids may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydrogen, hydroxyl, ketone, nitro, phosphate, sulfide, sulfinyl, sulfonyl, sulfonic acid, sulfonamide and thioketone. Dicarboxylic acids include, but are not limited to succinic acid, glutaric acid, adipic acid, suberic acid, sebacic acid, azelaic acid, maleic acid, phthalic acid, aspartic acid, glutamic acid, malonic acid, fumaric acid, (+)/(−)-malic acid, (+)/(−) tartaric acid, isophthalic acid, and terephthalic acid. Dicarboxylic acids further include carboxylic acid derivatives thereof, such as anhydrides, imides, hydrazides (for example, succinic anhydride and succinimide).

The term “ester” refers to the structure C(O)O—, C(O)ORⱼ, RₖC(O)O—Rⱼ, or RₖC(O)O—, where O is not bound to hydrogen, and Rⱼ and Rₖ can independently be selected from alkoxy, aryloxy, alkyl, alkenyl, alkynyl, amide, amino, aryl, arylalkyl, cycloalkyl, ether, haloalkyl, heteroaryl, and heterocyclyl. Rₖ can be a hydrogen, but Rⱼ cannot be hydrogen. The ester may be cyclic, for example the carbon atom and Rⱼ, the oxygen atom and Rₖ, or Rⱼ and Rₖ may be joined to form a 3- to 12-membered ring. Exemplary esters include, but are not limited to, alkyl esters wherein at least one of Rⱼ and Rₖ is alkyl, such as O—C(O) alkyl, C(O)—O-alkyl, and alkyl C(O)—O-alkyl. Exemplary esters also include aryl or heteoaryl esters, e.g. wherein at least one of Rⱼ and Rₖ is a heteroaryl group such as pyridine, pyridazine, pyrimidine and pyrazine, such as a nicotinate ester. Exemplary esters also include reverse esters having the structure RₖC(O)O—, where the oxygen is bound to the parent molecule. Exemplary reverse esters include succinate, D-argininate, L-argininate, L-lysinate and D-lysinate. Esters also include carboxylic acid anhydrides and acid halides.

The terms “halo” or “halogen” as used herein refer to F, Cl, Br, or I.

The term “haloalkyl” as used herein refers to an alkyl group substituted with one or more halogen atoms. “haloalkyls” also encompass alkenyl or alkynyl groups substituted with one or more halogen atoms.

The term “heteroaryl” as used herein refers to a mono-, bi-, or multi-cyclic, aromatic ring system containing one or more heteroatoms, for example 1 to 3 heteroatoms, such as nitrogen, oxygen, and sulfur. Heteroaryls can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heteroaryls can also be fused to non-aromatic rings. Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3)- and (1,2,4)-triazolyl, pyrazinyl, pyrimidilyl, tetrazolyl, furyl, thienyl, isoxazolyl, thiazolyl, furyl, phenyl, isoxazolyl, and oxazolyl. Exemplary heteroaryl groups include, but are not limited to, a monocyclic aromatic ring, wherein the ring comprises 2-5 carbon atoms and 1-3 heteroatoms, referred to herein as “(C₂-C₅) heteroaryl.”

The terms “heterocycle,” “heterocyclyl,” or “heterocyclic” as used herein refer to a saturated or unsaturated 3, 4, 5-, 6- or 7-membered ring containing one, two, or three heteroatoms independently selected from nitrogen, oxygen, and sulfur. Heterocycles can be aromatic (heteroaryls) or non-aromatic. Heterocycles can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heterocycles also include bicyclic, tricyclic, and tetracyclic groups in which any of the above heterocyclic rings is fused to one or two rings independently selected from aryls, cycloalkyls, and heterocycles. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl.

The terms “hydroxy” and “hydroxyl” as used herein refer to —OH.

The term “hydroxyalkyl” as used herein refers to a hydroxy attached to an alkyl group.

The term “hydroxyaryl” as used herein refers to a hydroxy attached to an aryl group.

The term “ketone” as used herein refers to the structure C(O)—Rₙ (such as acetyl, C(O)CH₃) or Rₙ—C(O)—Rₒ. The ketone can be attached to another group through Rₙ or Rₒ. Rₙ and Rₒ can be alkyl, alkenyl, alkynyl, cycloalkyl, heterocyclyl or aryl, or Rₙ and Rₒ can be joined to form a 3- to 12 membered ring.

The term “phenyl” as used herein refers to a 6-membered carbocyclic aromatic ring. The phenyl group can also be fused to a cyclohexane or cyclopentane ring. Phenyl can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, phosphate, sulfide, sulfinyl, sulfonyl, sulfonic acid, sulfonamide and thioketone.

The term “thioalkyl” as used herein refers to an alkyl group attached to a sulfur (S-alkyl).

“Alkyl,” “alkenyl,” “alkynyl”, “alkoxy”, “amino” and “amide” groups can be optionally substituted with or interrupted by or branched with at least one group selected from alkoxy, aryloxy, alkyl, alkenyl, alkynyl, amide, amino, aryl, arylalkyl, carbamate, carbonyl, carboxy, cyano, cycloalkyl, ester, ether, formyl, halogen, haloalkyl, heteroaryl, heterocyclyl, hydroxyl, ketone, phosphate, sulfide, sulfinyl, sulfonyl, sulfonic acid, sulfonamide, thioketone, ureido and N. The substituents may be branched to form a substituted or unsubstituted heterocycle or cycloalkyl.

As used herein, a suitable substitution on an optionally substituted substituent refers to a group that does not nullify the synthetic or pharmaceutical utility of the compounds of the present disclosure or the intermediates useful for preparing them. Examples of suitable substitutions include, but are not limited to: C₁-C₈ alkyl, C₂-C₈ alkenyl or alkynyl; C₆ aryl, 5- or 6-membered heteroaryl; C₃-C₇ cycloalkyl; C₁-C₈ alkoxy; C₆ aryloxy; CN; OH; oxo; halo, carboxy; amino, such as NH(C₁-C₈ alkyl), N(C₁-C₈ alkyl)₂, NH((C₆)aryl), or N((C₆)aryl)₂; formyl; ketones, such as CO(C₁-C₈ alkyl), —CO((C₆ aryl) esters, such as CO₂(C₁-C₈ alkyl) and CO₂(C₆ aryl). One of skill in art can readily choose a suitable substitution based on the stability and pharmacological and synthetic activity of the compound of the present disclosure.

The term “pharmaceutically acceptable composition” as used herein refers to a composition comprising at least one compound as disclosed herein formulated together with one or more pharmaceutically acceptable carriers.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions. The term “pharmaceutically acceptable composition” as used herein refers to a composition comprising at least one compound as disclosed herein formulated together with one or more pharmaceutically acceptable carriers.

The term “pharmaceutically acceptable prodrugs” as used herein represents those prodrugs of the compounds of the present invention that are, within the scope of sound medical judgment, suitable for use in contact with the tissues of humans and lower animals without undue toxicity, irritation, allergic response, commensurate with a reasonable benefit/risk ratio, and effective for their intended use, as well as the zwitterionic forms, where possible, of the compounds of Formula I. A discussion is provided in Higuchi et al., “Prodrugs as Novel Delivery Systems,” ACS Symposium Series, Vol. 14, and in Roche, E. B., ed. Bioreversible Carriers in Drug Design, American Pharmaceutical Association and Pergamon Press, 1987, both of which are incorporated herein by reference.

The term “pharmaceutically acceptable salt(s)” refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, matate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene -bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

The compounds of Formula I or Ia may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly.

Individual stereoisomers of compounds for use in the methods of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

Geometric isomers can also exist in the compounds of Formula I or Ia. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the E and Z isomers.

Substituents around a carbon-carbon double bond alternatively can be referred to as “cis” or “trans,” where “cis” represents substituents on the same side of the double bond and “trans” represents substituents on opposite sides of the double bond. The arrangements of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The compounds of Formula I disclosed herein may exist as tautomers and both tautomeric forms are intended to be encompassed by the scope of the invention, even though only one tautomeric structure is depicted.

As used herein, the term “dipeptidyl peptidase 4 inhibitor” or “DPP-4 inhibitor” refers a substance, such as a small molecule organic chemistry compounds (≤1 kDa) or a large biomolecule such as a peptide (e.g., a soluble peptide), protein (e.g., an antibody), nucleic acid (e.g., siRNA) or a conjugate combining any two or more of the foregoing, that possesses the activity of inhibiting the enzyme dipeptidyl peptidase 4 (DPP-4). Non-limiting examples of DPP-4 inhibitors include sitagliptin, saxagliptin, linagliptin, alopgliptin, vildagliptin, anagliptin, trelagliptin, omarigliptin, evogliptin, gosogliptin, gemigliptin, teneligliptin, or dutogliptin, or a pharmaceutically acceptable salt of any of the foregoing.

As used herein, “treatment” or “treating” refers to an amelioration of a disease or disorder, or at least one discernible symptom thereof. In another embodiment, “treatment” or “treating” refers to an amelioration of at least one measurable physical parameter, not necessarily discernible by the patient. In yet another embodiment, “treatment” or “treating” refers to reducing the progression of a disease or disorder, either physically, e.g., stabilization of a discernible symptom, physiologically, e.g., stabilization of a physical parameter, or both. In yet another embodiment, “treatment” or “treating” refers to delaying the onset or progression of a disease or disorder. For example, treating a cholesterol disorder may comprise decreasing blood cholesterol levels.

As used herein, “prevention” or “preventing” refers to a reduction of the risk of acquiring a given disease or disorder or a symptom of a given disease or disorder.

The term “narrowly defined MACE” is defined as a single composite endpoint of Cardiovascular (CV) death, non-fatal Myocardial infarction, or stroke.

The term “broadly defined MACE” is defined as a single composite endpoint of Cardiovascular (CV) death, non-fatal Myocardial infarction, hospitalization for CVD events, or stroke.

As used herein, “cardiovascular disease events” or “CVD events” are physical manifestations of cardiovascular-related disorders, and include events such as stroke, non-fatal myocardial infarction, cardiovascular death, and hospitalization for CVD events and congestive heart failure. As used herein, “hospitalization for CVD events” is defined as hospitalization for unstable angina, symptoms of progressive obstructive coronary disease, emergency revascularization procedures at any time, or urgent revascularization procedures ≥30 days after the index events prior to randomization. In some embodiments, “hospitalization for CVD events” includes hospitalization for physical manifestations of cardiovascular-related disorders, including congestive heart failure. In one embodiment, the hospitalization for CVD events is hospitalization for congestive heart failure.

As used herein, “cardiovascular-related disorders” include: cardiovascular death, non-fatal myocardial infarction, stroke, hospitalization for CVD events which includes unstable angina, symptoms of progressive obstructive coronary disease, emergency revascularization procedures at any time, or urgent revascularization procedures ≥30 days after index event, and congestive heart failure.

As used herein, a “recent acute coronary syndrome” or “recent ACS” refers to a condition or a range of conditions associated with sudden, reduced blood flow to the heart that occurs in a subject at 7-90 days prior to the subject being treated with at least one substance selected from statin (high-intensity statin treatment or maximum tolerated statin treatment), apabetalone, and a DPP-4 inhibitor as defined herein. One such condition is a heart attack or myocardial infarction, when cell death results in damaged or destroyed heart tissue. Another such condition is when the sudden, reduced blood flow to the heart causes no cell death, but changes how the heart works and is a sign of a high risk of heart attack. Signs and symptoms of ACS, which usually begin abruptly, include but are not limited to: chest pain (angina) or discomfort, often described as aching, pressure, tightness or burning; pain spreading from the chest to the shoulders, arm, upper abdomen, back, neck, or jaw; nausea or vomiting; indigestion; shortness of breath (dyspnea); sudden, heavy sweating (diaphoresis); lightheadedness, dizziness, or fainting; unusual or unexplained fatigue; and feeling restless or apprehensive.

### Exemplary Embodiments of the Invention

In one embodiment, the present invention provides methods of treating and/or preventing major adverse cardiovascular events (MACE), including non-fatal myocardial infarction, CV death, stroke, and hospitalization for CVD events, by administering to a subject in need thereof, a combination of a dipeptidyl peptidase 4 (DPP-4) inhibitor and a compound of Formula I or a stereoisomer, tautomer, pharmaceutically acceptable salt, or hydrate thereof, wherein:

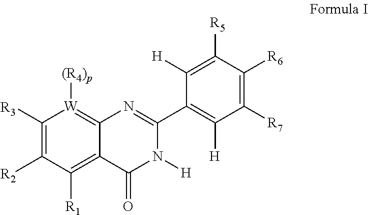

or a stereoisomer, tautomer, pharmaceutically acceptable salt, or hydrate thereof,

wherein:

R₁ and R₃ are each independently selected from alkoxy, alkyl, amino, halogen, and hydrogen;

R₂ is selected from alkoxy, alkyl, alkenyl, alkynyl, amide, amino, halogen, and hydrogen;

R₅ and R₇ are each independently selected from alkyl, alkoxy, amino, halogen, and hydrogen;

R₆ is selected from amino, amide, alkyl, hydrogen, hydroxyl, piperazinyl, and alkoxy;

W is selected from C and N, wherein if W is N, then p is 0 or 1, and if W is C, then p is 1; and

for W—(R₄)ₚ, W is C, p is 1 and R₄ is H, or W is N and p is 0.

In one embodiment, the compound of Formula I is 2-(4-(2-hydroxyethoxy)-3,5-dimethylphenyl)-5,7-dimethoxyquinazolin-4(3H)-one (RVX-208 or RVX000222) or a pharmaceutically acceptable salt thereof.

In one embodiment, the DPP-4 inhibitor is selected from sitagliptin, saxagliptin, linagliptin, alopgliptin, vildagliptin, anagliptin, trelagliptin, omarigliptin, evogliptin, gosogliptin, gemigliptin, teneligliptin, or dutogliptin.

In one embodiment, the MACE endpoint is narrowly defined as a single composite endpoint of cardiovascular (CV) death, non-fatal myocardial infarction, or stroke.

In one embodiment, the MACE endpoint is broadly defined as a single composite endpoint of cardiovascular (CV) death, non-fatal myocardial infarction, hospitalization for CVD events, or stroke.

In one embodiment, the method for treating and/or preventing any individual component of MACE, including cardiovascular (CV) death, non-fatal myocardial infarction, hospitalization for CVD events, or stroke by administrating to a subject in need thereof, a dipeptidyl peptidase 4 (DPP-4) inhibitor and a Compound of Formula Ia or a stereoisomer, tautomer, pharmaceutically acceptable salt, or hydrate thereof, wherein:

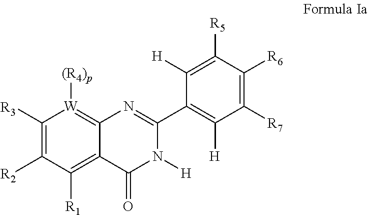

R₁ and R₃ are each independently selected from alkoxy, alkyl, and hydrogen;

R₂ is selected from alkoxy, alkyl, and hydrogen;

R₅ and R₇ are each independently selected from alkyl, alkoxy, amino, halogen, and hydrogen;

R₆ is selected from alkyl, hydroxyl, and alkoxy;

W is selected from C and N, wherein if W is N, then p is 0 or 1, and if W is C, then p is 1; and

for W—(R₄)ₚ, W is C, p is 1 and R₄ is H, or W is N and p is 0.

In one embodiment, the compound of Formula I is administered simultaneously with the DPP-4 inhibitor.

In one embodiment, the Compound of Formula I is administered sequentially with the DPP-4 inhibitor.

In one embodiment, the Compound of Formula I is administered in a single pharmaceutical composition with the DPP-4 inhibitor.

In one embodiment, the Compound of Formula I and the DPP-4 inhibitor are administered as separate compositions.

In one embodiments, a subject in need thereof is given 200 mg daily of 2-(4-(2-hydroxyethoxy)-3,5-dimethylphenyl)-5,7-dimethoxyquinazolin-4(3H)-one or an equivalent amount of a pharmaceutically acceptable salt thereof.

In one embodiment, a subject in need thereof is given 100 mg of 2-(4-(2-hydroxyethoxy)-3,5-dimethylphenyl)-5,7-dimethoxyquinazolin-4(3H)-one or an equivalent amount of a pharmaceutically acceptable salt thereof twice daily.

In one embodiment, the subject is a human.

In one embodiment, the subject is a human with type 2 diabetes and low HDL cholesterol (below 40 mg/dL for males and below 45 mg/dL for females) and a recent acute coronary syndrome (ACS).

In one embodiment, the subject is a human with type 2 diabetes.

In one embodiment, the subject is a human with low HDL cholesterol (i.e., below 40 mg/dL for males and below 45 mg/dL for females).

In one embodiment, the subject is a human with a recent ACS.

In one embodiment, the subject is a human on statin therapy. In one embodiment, the subject is a human on high intensity or maximum tolerated statin therapy. In one embodiment, the high intensity statin treatment or therapy refers to a daily dose of at least 20 mg, or at least 40 mg, or 20-80 mg, or 20-40 mg, or 40-80 mg. In one embodiment, the maximum tolerated statin treatment or therapy refers to a daily lose of at least 40 mg, or 40 mg-80 mg, or 80 mg. In one embodiment, the subject is on rosuvastatin therapy. In one embodiment, the subject is on atorvastatin therapy.

### REFERENCES

Cannon, C. P., Blazing, M. A., Giugliano, R. P., et al. (2015) Ezetimibe added to statin therapy after acute coronary syndromes. N Engl J Med, 372(25), 2387-97.
Schwartz, G. G., Olsson, A. G. & Barter, P. J. (2013) Dalcetrapib in patients with an acute coronary syndrome. N Engl J Med, 368(9), 869-70.
Schwartz, G. G., Steg, P. G., Szarek, M., et al. (2018) Alirocumab and cardiovascular outcomes after acute coronary syndrome. N Engl J Med, 379(22), 2097-107.
Thornberry, N. A., and Gallwitz, B. (2009) Mechanism of action of inhibitors of dipeptidyl-peptidase-4 (DPP-4). Best Pract Res Clin Endocrinol Metab, 23(4),479-86.
Rosenstock, J., Perkovic, V., Johansen, O. E., et al. (2019) Effect of Linagliptin vs Placebo on Major Cardiovascular Events in Adults with Type 2 Diabetes and High Cardiovascular and Renal Risk. JAMA, 321(1), 69-79.
Green, J. B., Bethel, M. A., Armstrong, P. W., et al. (2015) Effect of Sitagliptin on Cardiovascular Outcomes in Type 2 Diabetes. N Engl J Med, 373, 232-42.
Scirica, B. M., Bhatt, D. L., Braunwald, E., et al. (2013) Saxagliptin and Cardiovascular Outcomes in Patients with Type 2 Diabetes Mellitus. N Engl J Med, 369, 1317-26.
White, W. B., Cannon, C. P., Heller, S. R., et al. (2013) Alogliptin after Acute Coronary Syndrome in Patients with Type 2 Diabetes. N Engl J Med, 369, 1327-35.
Karagiannis, T., Bekiari, E., Boura, P., et al. (2015) Cardiovascular Risk with DPP-4 Inhibitors: Latest Evidence and Clinical Implications. Ther Adv Drug Saf, 7(2), 36-38.

### EXAMPLES

### Example 1

### Clinical Development

Apabetalone (RVX-208) was evaluated in a recently completed clinical Phase 3 trial (BETonMACE; NCT02586155) for the effect on MACE in type 2 diabetes patients with low HDL cholesterol (below 40 mg/dL for males and below 45 mg/dL for females) and a recent acute coronary syndrome (ACS). All patients received high intensity statin treatment, which was 20-40 mg daily or a maximum daily dose of 40 mg for rosuvastatin or 40-80 mg daily or a maximum daily dose of 80 mg for atorvastatin.

Patients (n=2425) with ACS in the preceding 7 to 90 days, with type 2 diabetes and low HDL cholesterol (≤40 mg/dl for men, ≤45 mg/dl for women), receiving intensive or maximum-tolerated therapy with atorvastatin or rosuvastatin, were assigned in double-blind fashion to receive apabetalone 100 mg orally twice daily or matching placebo. Baseline characteristics include female sex (25%), myocardial infarction as index ACS event (74%), coronary revascularization for index ACS (76%), treatment with dual anti-platelet therapy (87%) and renin-angiotensin system inhibitors (91%), median LDL cholesterol 65 mg per deciliter, and median HbA1c 7.3%. The primary efficacy measure is time to first occurrence of cardiovascular death, non-fatal myocardial infarction, or stroke. Assumptions include a primary event rate of 7% per annum in the placebo group and median follow-up of 1.5 years. Patients were followed until at least 250 primary endpoint events had occurred, providing 80% power to detect a 30% reduction in the primary endpoint with apabetalone.

### Example 2

### Post-Hoc Analysis

In the BETonMACE clinical study, a total of N=336 patients (N=169 in apabetalone treatment group and N=167 in placebo treatment group) were administered a DPP-4 inhibitor (selected from alogliptin, linagliptin, saxagliptin, sitagliptin, teneligliptin and vildagliptin) in addition to RVX-208 with specified statin therapy (atorvastatin and rosuvastatin) and other guideline-defined treatments. Patients who were randomized and received at least one dose of DPP-4 inhibitor treatment prior to the date of the first incidence of event were censored as a MACE event at the date of the confirmed event. Those patients who received at least one dose of DPP-4 inhibitor treatment after the date of the first incidence of event were censored as non-MACE events and the date of last contact was used as the censoring date. For all patients who did not receive DPP-4 inhibitor treatment during the study, the time to first event was calculated using randomization date and date of the confirmed event, or date of last contact for censored subjects.

The distributions of the endpoints within the apabetalone and placebo groups were compared using a two-sided log-rank test (LRT) with an alpha=0.05 level of significance. The cumulative incidence is shown as 1-KM (Kaplan-Meier) estimate for event rate.

Narrowly Defined MACE

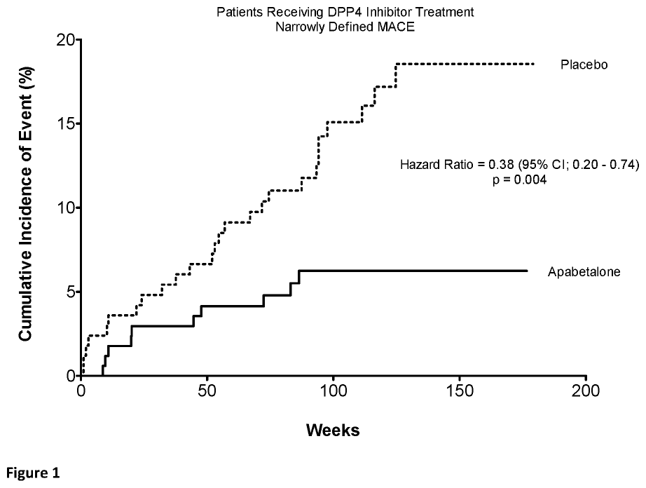

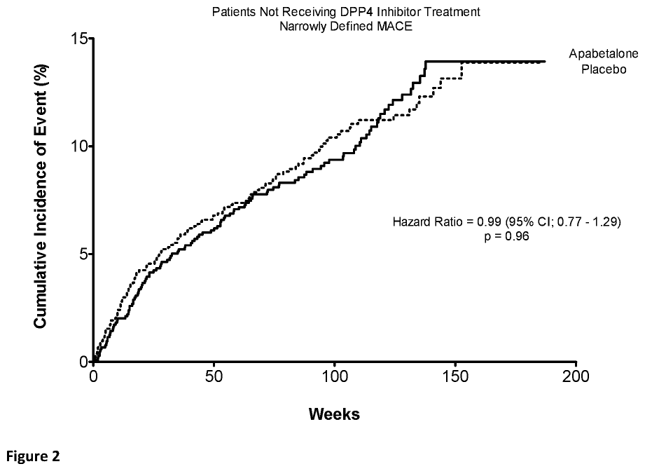

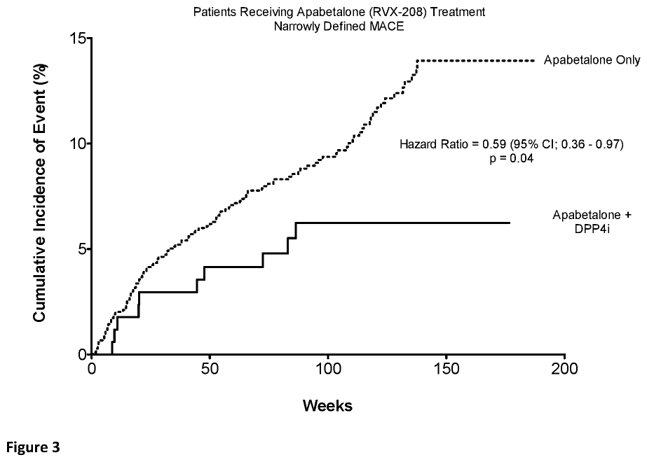

FIGS. 1-3 each compare the cumulative incidence of narrowly defined MACE (i.e., as a single composite endpoint of multiple primary end points defined as cardiovascular death, non-fatal myocardial infarction, or stroke) between two groups of patients, a test group and a control group, which are described as follows:
i. patients receiving DPP-4 inhibitor treatment: administered with apabetalone (test) or a placebo (control) (FIG. 1);
ii. patients not receiving DPP-4 inhibitor treatment: administered with apabetalone (test) or placebo (control) (FIG. 2); and
iii. patients receiving apabetalone treatment: administered with a DPP-4 inhibitor (test) or not administered with a DPP-4 inhibitor (control) (FIG. 3).

In FIG. 1, where the patients were treated with a DPP-4 inhibitor and received either apabetalone or a placebo, there were a total of 36 primary end points: 10 (5.9%) in the apabetalone group and 26 (15.6%) in the placebo group, representing a Kaplan-Meier estimated event rate of 4.8% in the apabetalone group and 11.8% in the placebo group at 18 months. This means that at 18 months, patients treated with only the DPP-4 inhibitor had an estimated narrowly defined MACE event rate at 11.8% but when patients were treated with the combination of apabetalone and a DPP-4 inhibitor, the estimated narrowly defined MACE event rate was reduced by nearly 60% at 4.8%. As depicted in FIG. 1, combining apabetalone with a DPP-4 inhibitor significantly reduced the composite end point of narrowly defined MACE compared to treatment with the DPP-4 inhibitor alone, specifically by reducing the number of patients having a narrowly defined MACE event at any given time by 62% (Hazard Ratio [HR], 0.38; 95% CI, 0.20-0.74; P=0.004).

In FIG. 2, where the patients were not treated with a DPP-4 inhibitor and received either apabetalone or a placebo, there were a total of 228 primary end points: 114 (10.9%) in the apabetalone group and 114 (11.0%) in the placebo group, representing a Kaplan-Meier estimated event rate of 8.0% in the apabetalone group and 8.3% in the placebo group at 18 months. This means that at 18 months, patients treated with only apabetalone had an estimated narrowly defined MACE event rate of 10.9% while patients that were not treated with apabetalone or a DPP-4 inhibitor had an estimated narrowly defined MACE event rate of 11.0%. As depicted in FIG. 2, apabetalone monotherapy did not reduce the composite end point of narrowly defined MACE compared to non-treatment (Hazard Ratio [HR], 0.99; 95% CI, 0.77-1.29; P=0.96).

As depicted in FIG. 3, patients treated with the combination of apabetalone and a DPP-4 inhibitor, when compared to patients treated with apabetalone alone, exhibited a significant hazard ratio of 0.59 (95% CI, 0.36-0.97; P=0.04) for the composite end point of narrowly defined MACE. This means that the combination of apabetalone and a DPP-4 inhibitor reduced the number of patients having a narrowly defined MACE event at any given time by 41%, compared to treatment with apabetalone alone.

In conclusion, apabetalone monotherapy did not reduce the number of patients having a narrowly defined MACE event at any given time compared to non-treatment (see FIG. 2). Additionally, as established in the background of this disclosure, no DPP-4 inhibitor has been shown to have any effect in reducing MACE. Thus, it was unexpected that a combination therapy of apabetalone and a DPP-4 inhibitor, each ineffective as monotherapies, results in any reduction of the number of patients having a narrowly defined MACE event at any given time, much less a significant reduction of 62% compared to DPP-4 inhibitor monotherapy, or 59% compared to apabetalone monotherapy.

Broadly Defined MACE

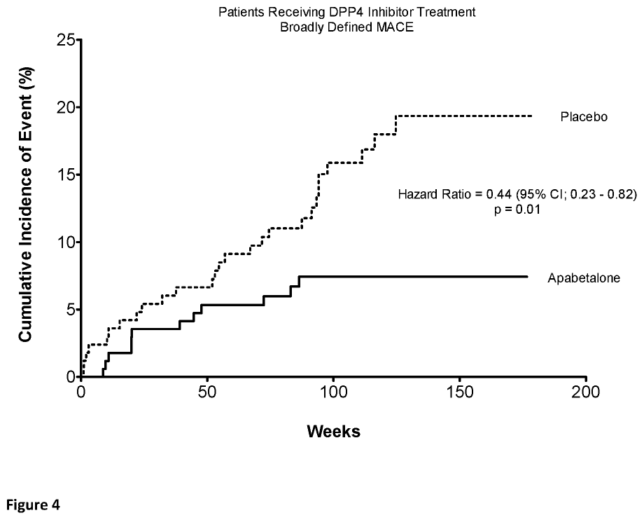

FIGS. 4-6 each compare the cumulative incidence of broadly defined MACE (i.e., as a single composite endpoint of multiple primary end points defined as cardiovascular death, non-fatal myocardial infarction, stroke, or hospitalization for cardiovascular diseases (CVD)) between the same two groups of patients as described above for FIGS. 1-3.

In FIG. 4, where the patients were treated with a DPP-4 inhibitor and received either apabetalone or a placebo, it can be seen that combining apabetalone with a DPP-4 inhibitor reduced the composite end point of broadly defined MACE compared to treatment with the DPP-4 inhibitor alone (with trending statistical significance), specifically by reducing the number of patients having a broadly defined MACE event at any given time by 56% (Hazard Ratio [HR], 0.44; 95% CI, 0.23-0.82; P=0.01).

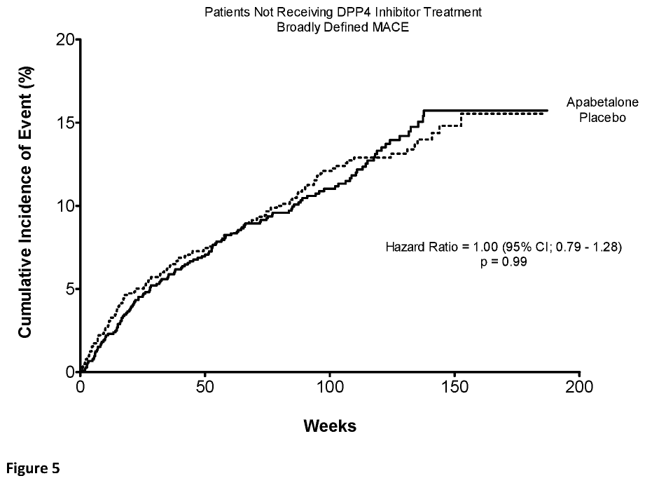

In FIG. 5, where the patients were not treated with a DPP-4 inhibitor but received either apabetalone or a placebo, it can be seen that apabetalone monotherapy did not reduce the composite end point of broadly defined MACE compared to non-treatment (Hazard Ratio [HR], 1.00; 95% CI, 0.79-1.28; P=0.99).

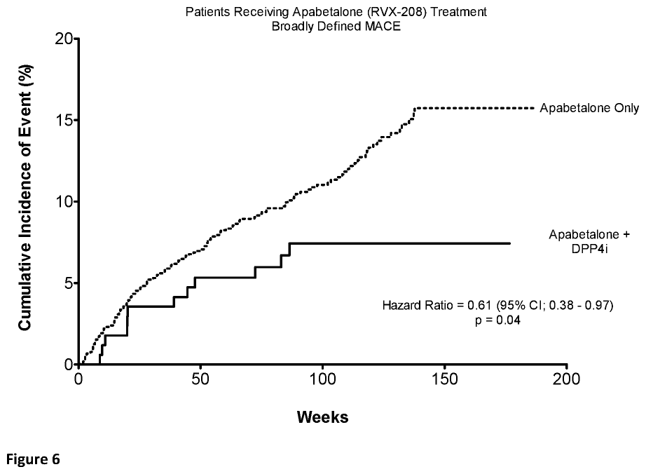

As depicted in FIG. 6, patients treated with the combination of apabetalone and a DPP-4 inhibitor, when compared to patients treated with apabetalone alone, exhibited a significant hazard ratio of 0.61 (95% CI, 0.38-0.97; P=0.04) for the composite end point of broadly defined MACE. This means that the combination of apabetalone and a DPP-4 inhibitor reduced the number of patients having a broadly defined MACE event at any given time by 39%, compared to treatment with apabetalone alone.

In conclusion, apabetalone monotherapy did not reduce the number of patients having a broadly defined MACE event at any given time compared to non-treatment (see FIG. 5). Additionally, as established in the background of this disclosure, no DPP-4 inhibitor has been shown to have any effect in reducing MACE. Thus, it was unexpected that a combination therapy of apabetalone and a DPP-4 inhibitor, each ineffective as monotherapies, results in any reduction of the number of patients having a broadly defined MACE event at any given time, much less a significant reduction of 56% compared to DPP-4 inhibitor monotherapy, or 39% compared to apabetalone monotherapy.

Non-Fatal Myocardial Infarction

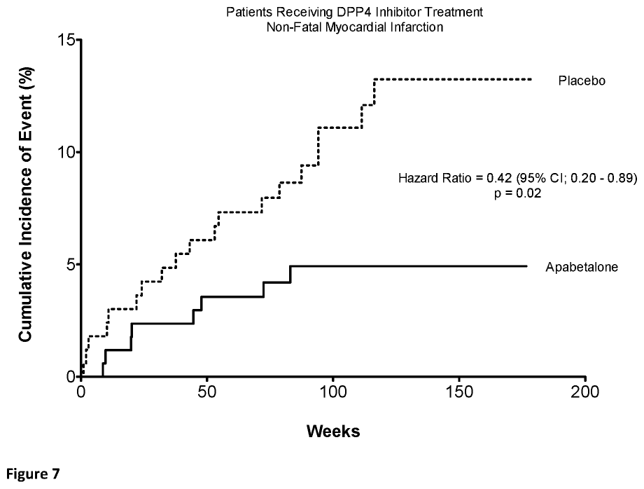

FIGS. 7-9 each compare the cumulative incidence of non-fatal myocardial infarction between the same two groups of patients as described as described above for FIGS. 1-3.

In FIG. 7, where the patients were treated with a DPP-4 inhibitor and received either apabetalone or a placebo, it can be seen that combining apabetalone with a DPP-4 inhibitor significantly reduced for the end point of non-fatal myocardial infarction compared to treatment with the DPP-4 inhibitor alone, specifically by reducing the number of patients having a non-fatal myocardial infarction event at any given time by 58% (Hazard Ratio [HR], 0.42; 95% CI, 0.20-0.89; P=0.02).

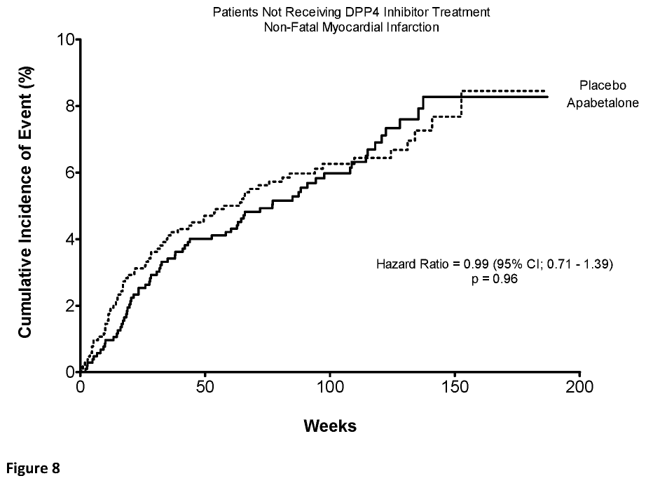

In FIG. 8, where the patients were not treated with a DPP-4 inhibitor but received either apabetalone or a placebo, it can be seen that apabetalone monotherapy did not reduce the end point of non-fatal myocardial infarction compared to non-treatment (Hazard Ratio [HR], 0.99; 95% CI, 0.71-1.39; P=0.96).

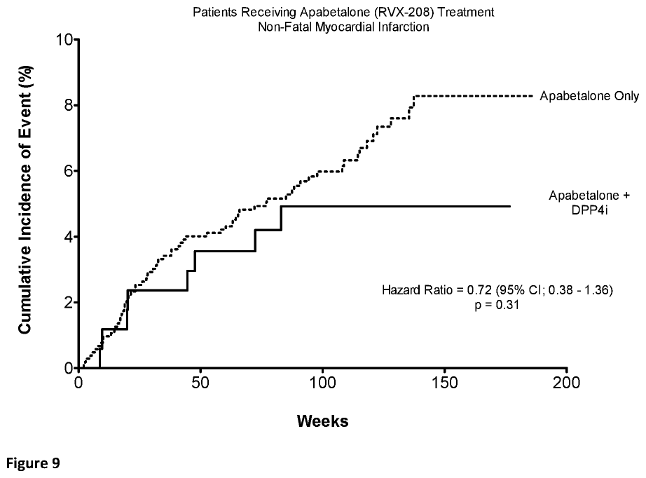

As depicted in FIG. 9, patients treated with the combination of apabetalone and a DPP-4 inhibitor, when compared to patients treated with apabetalone alone, exhibited a hazard ratio of 0.72 (95% CI, 0.38-1.06; P=0.31) for the end point of non-fatal myocardial infarction. This means that the combination of apabetalone and a DPP-4 inhibitor reduced the number of patients having a non-fatal myocardial infarction event at any given time by 28%, compared to treatment with apabetalone alone.

In conclusion, apabetalone monotherapy did not reduce the number of patients having a non-fatal myocardial infarction event at any given time compared to non-treatment (see FIG. 8). Additionally, as established in the background of this disclosure, no DPP-4 inhibitor has been shown to have any effect in reducing MACE. Thus, it was unexpected that a combination therapy of apabetalone and a DPP-4 inhibitor, each ineffective as monotherapies, results in any reduction of the number of patients having a non-fatal myocardial infarction event at any given time, much less a significant reduction of 58% compared to DPP-4 inhibitor monotherapy, or a reduction of 28% compared to apabetalone monotherapy.

Cardiovascular Death

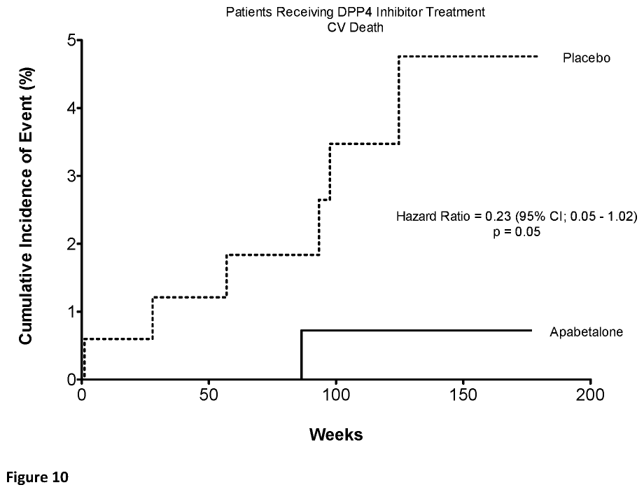

FIGS. 10-12 each compare the cumulative incidence of cardiovascular death between the same two groups of patients as described as described above for FIGS. 1-3.

In FIG. 10, where the patients were treated with a DPP-4 inhibitor and received either apabetalone or a placebo, it can be seen that combining apabetalone with a DPP-4 inhibitor significantly reduced the end point of cardiovascular death compared to treatment with the DPP-4 inhibitor alone, specifically by reducing the number of patients having a cardiovascular death event at any given time by 77% (Hazard Ratio [HR], 0.23; 95% CI, 0.05-1.02; P=0.05).

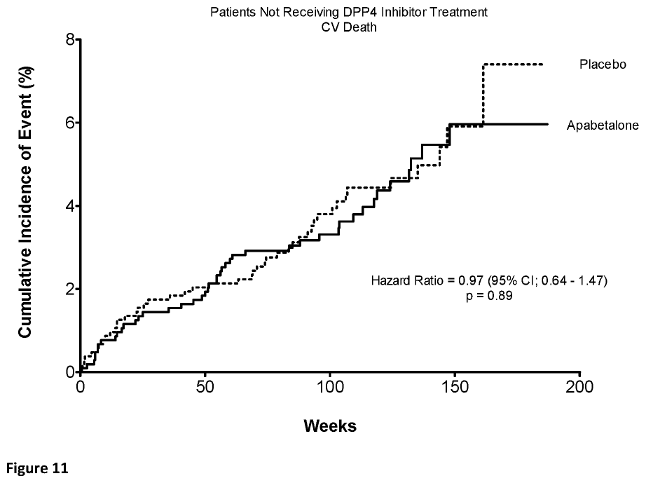

In FIG. 11, where the patients were not treated with a DPP-4 inhibitor but received either apabetalone or a placebo, it can be seen that apabetalone monotherapy did not reduce the end point of cardiovascular death compared to non-treatment (Hazard Ratio [HR], 0.97; 95% CI, 0.64-1.47; P=0.89).

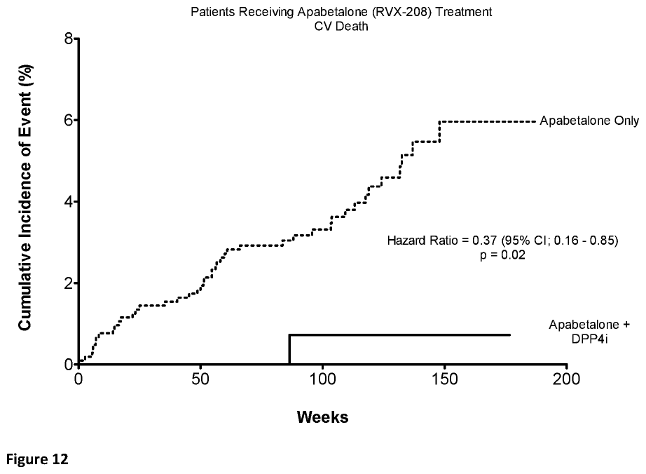

As depicted in FIG. 12, patients treated with the combination of apabetalone and a DPP-4 inhibitor, when compared to patients treated with apabetalone alone, exhibited a significant hazard ratio of 0.37 (95% CI, 0.16-0.85; P=0.02) for the end point of cardiovascular death. This means that the combination of apabetalone and a DPP-4 inhibitor reduces the number of patients having a cardiovascular death event at any given time by 63%, compared to treatment with apabetalone alone.

In conclusion, apabetalone monotherapy did not reduce the number of patients having a cardiovascular death event at any given time compared to non-treatment (see FIG. 11). Additionally, as established in the background of this disclosure, no DPP-4 inhibitor has been shown to have any effect in reducing MACE. Thus, it was unexpected that a combination therapy of apabetalone and a DPP-4 inhibitor, each ineffective as monotherapies, results in any reduction of the number of patients having a cardiovascular death event at any given time, much less a significant reduction of 77% compared to DPP-4 inhibitor monotherapy, 63% compared to apabetalone monotherapy.

Hospitalization for Congestive Heart Failure

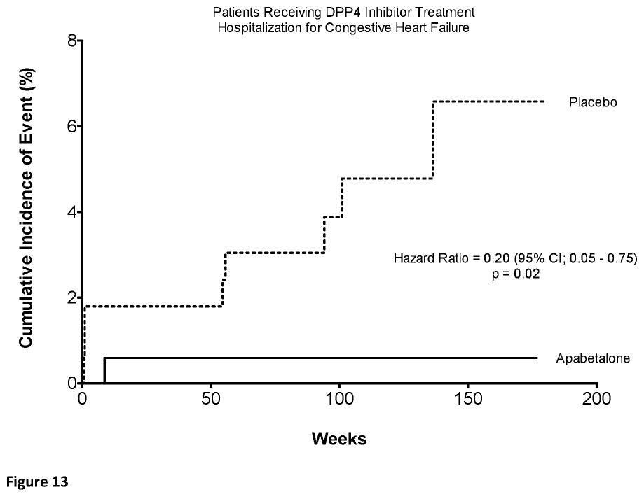

FIGS. 13-15 each compare the cumulative incidence of hospitalization for congestive heart failure between the same two groups of patients as described as described above for FIGS. 1-3.

In FIG. 13, where the patients were treated with a DPP-4 inhibitor and received either apabetalone or a placebo, it can be seen that combining apabetalone with an DPP-4 inhibitor significantly reduced the end point of hospitalization for congestive heart failure compared to treatment with the DPP-4 inhibitor alone, specifically by reducing the number of patients having a hospitalization for congestive heart failure event at any given time by 80% (Hazard Ratio [HR], 0.20; 95% CI, 0.05-0.75; P =0.02).

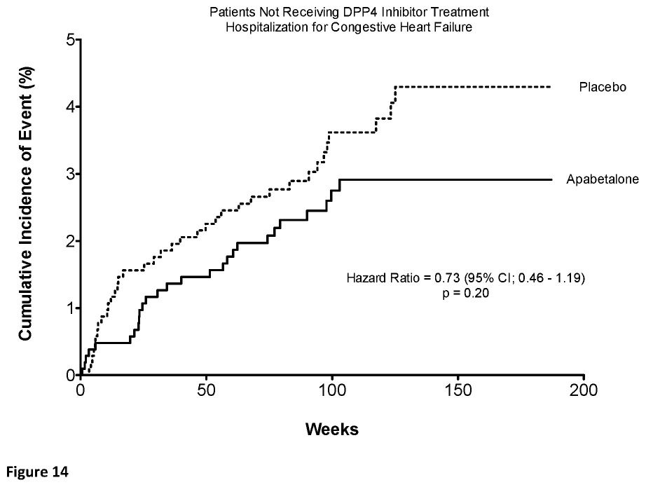

In FIG. 14, where the patients were not treated with a DPP-4 inhibitor but received either apabetalone or a placebo, it can be seen that apabetalone monotherapy reduced the end point of hospitalization for congestive heart failure compared to non-treatment, specifically by reducing the number of patients having a hospitalization for congestive heart failure event at any given time by 27% (Hazard Ratio [HR], 0.73; 95% CI, 0.46-1.19; P=0.20),

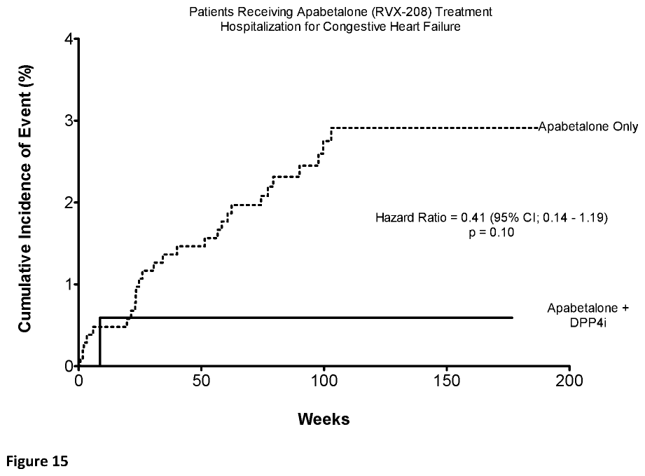

As depicted in FIG. 15, patients treated with the combination of apabetalone and a DPP-4 inhibitor, when compared to patients treated with apabetalone alone, exhibited a hazard ratio of 0.41 (95% CI, 0.14-1.19; P=0.10) for the end point of hospitalization for congestive heart failure. This means that the combination of apabetalone and a DPP-4 inhibitor reduced the number of patients having a hospitalization for congestive heart failure event at any given time by 59%, compared to treatment with apabetalone alone.

In conclusion, apabetalone monotherapy was able to reduce the number of patients having a hospitalization for congestive heart failure event at any given time by 27% compared to patients receiving only the placebo (see FIG. 14). Additionally, as established in the background of this disclosure, no DPP-4 inhibitor has been shown to have any effect in reducing MACE. Thus, it was unexpected that a combination therapy of apabetalone and a DPP-4 inhibitor, when the latter is ineffective as a monotherapy, results in a significant reduction of 80% at any given time in the number of patients having a hospitalization for congestive heart failure compared to DPP-4 inhibitor monotherapy, or a reduction of 59% compared to apabetalone monotherapy.

1. A method for treating and/or preventing major adverse cardiovascular events (MACE) comprising administering to a subject in need thereof, a dipeptidyl peptidase 4 (DPP-4) inhibitor and a compound of Formula I or a stereoisomer, tautomer, pharmaceutically acceptable salt, or hydrate thereof, wherein: 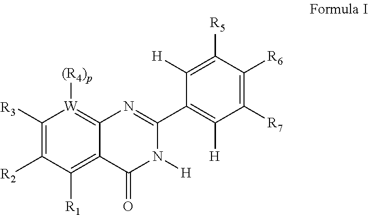
R₁ and R₃ are each independently selected from alkoxy, alkyl, amino, halogen, and hydrogen;
R₂ is selected from alkoxy, alkyl, alkenyl, alkynyl, amide, amino, halogen, and hydrogen;
R₅ and R₇ are each independently selected from alkyl, alkoxy, amino, halogen, and hydrogen;
R₆ is selected from amino, amide, alkyl, hydrogen, hydroxyl, piperazinyl, and alkoxy;
W is selected from C and N, wherein if W is N, then p is 0 or 1, and if W is C, then p is 1; and
for W—(R₄)ₚ, W is C, p is 1 and R₄ is H, or W is N and p is 0.

2. A method for treating and/or preventing any individual component of MACE comprising administrating to a subject in need thereof, a dipeptidyl peptidase 4 (DPP-4) inhibitor and a compound of Formula I or a stereoisomer, tautomer, pharmaceutically acceptable salt, or hydrate thereof, wherein: 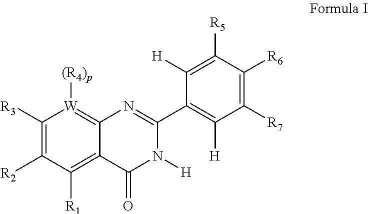
R₁ and R₃ are each independently selected from alkoxy, alkyl, amino, halogen, and hydrogen;
R₂ is selected from alkoxy, alkyl, alkenyl, alkynyl, amide, amino, halogen, and hydrogen;
R₅ and R₇ are each independently selected from alkyl, alkoxy, amino, halogen, and hydrogen;
R₆ is selected from amino, amide, alkyl, hydrogen, hydroxyl, piperazinyl, and alkoxy;
W is selected from C and N, wherein if W is N, then p is 0 or 1, and if W is C, then p is 1; and
for W—(R₄)ₚ, W is C, p is 1 and R₄ is H, or W is N and p is 0.

3. The method of claim 1 or claim 2, wherein the compound of Formula I is selected from compounds of Formula Ia: 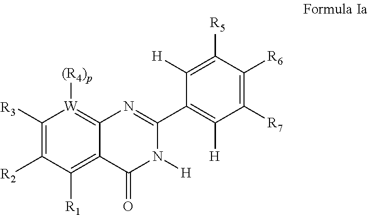
or a stereoisomer, tautomer, pharmaceutically acceptable salt, or hydrate thereof,
wherein:
R₁ and R₃ are each independently selected from alkoxy, alkyl, and hydrogen;
R₂ is selected from alkoxy, alkyl, and hydrogen;
R₅ and R₇ are each independently selected from alkyl, alkoxy, and hydrogen;
R₆ is selected from alkyl, hydroxyl, and alkoxy;
W is selected from C and N, wherein if W is N, then p is 0 or 1, and if W is C, then p is 1; and
for W—(R₄)ₚ, W is C, p is 1 and R₄ is H, or W is N and p is 0.

4. The method of any one of claims 1 to 3, wherein the compound of Formula I or Ia is 2-(4-(2-hydroxyethoxy)-3,5-dimethylphenyl)-5,7-dimethoxyquinazolin-4(3H)-one (RVX-208 or RVX000222) or a pharmaceutically acceptable salt thereof.

5. The method according to any one claim of claims 1 to 4, comprising administering a daily dose of 200 mg of 2-(4-(2-hydroxyethoxy)-3,5-dimethylphenyl)-5,7-dimethoxyquinazolin-4(3H)-one or an equivalent amount of a pharmaceutically acceptable salt thereof to a subject in need thereof.

6. The method of claim 5, wherein a subject in need thereof is administered 100 mg of 2-(4-(2-hydroxyethoxy)-3,5-dimethylphenyl)-5,7-dimethoxyquinazolin-4(3H)-one or an equivalent amount of a pharmaceutically acceptable salt thereof twice daily.

7. The method according to any one claim of claims 1 to 6, wherein the DPP-4 inhibitor is selected from alogliptin, linagliptin, saxagliptin, sitagliptin, teneligliptin and vildagliptin.

8. The method according to any one claim of claims 1 to 7, wherein the subject is a human.

9. The method according to any one claim of claims 1 to 8, wherein the subject is a human with type 2 diabetes and low HDL cholesterol (below 40 mg/dL for males and below 45 mg/dL for females) and recent acute coronary syndrome (ACS).

10. The method according to any one claim of claims 1-9, wherein the subject is on statin therapy.

11. The method according to any one of claims 1-10, wherein the MACE is selected from non-fatal myocardial infarction, cardiovascular death, stroke, and hospitalization for cardiovascular disease events.

12. The method according to claim 11, wherein the cardiovascular disease event is congestive heart failure.

13. The method according to claim 11, wherein the hospitalization for cardiovascular disease events is hospitalization for congestive heart failure.

14. The method according to any one of claims 1-10, wherein the MACE is selected from non-fatal myocardial infarction, cardiovascular death, and stroke.
